(19) United States
(12) Patent Application Publication
(10) Pub. No.: US 2009/0023209 A1
(43) Pub. Date: Jan. 22, 2009

(54) SOLUTION FOR THE INDEFINITE MAINTENANCE OF NUCLEIC ACIDS IN THE CELL OF ORIGIN THEREOF

(76) Inventors: Susana Dunner Boxberger, Madrid (ES); Javier Canon Ferreras, Madrid (ES)

Correspondence Address: SCHIFF HARDIN, LLP;PATENT DEPARTMENT, 6600 SEARS TOWER, CHICAGO, IL, 60606-6473, US

(21) Appl. No.: 11/576,747

(22) PCT Filed: Oct. 4, 2005
(86) PCT No.: PCT/ES05/00531

§ 371 Date: Aug. 14, 2008

(30) Foreign Application Priority Data
Oct. 5, 2004 (ES) P200402365

(51) Int. Cl. C12N 5/06; C12N 1/20

(52) U.S. Cl. 435/374; 435/260

(57) ABSTRACT

The invention relates to a product having characteristics which enable a biological sample obtained from an individual to be maintained under conditions such that the quality of nucleic acids later extracted is not affected, thereby guaranteeing the integrity of the sample regardless of the time that elapses between the sample being obtained and the nucleic acid being extracted. A solid (organic) or liquid (blood, semen, cell culture) biological sample from a eukaryote or prokaryote is mixed in a solution comprised of a mixture of trisodium ethylenediaminetetraacetic acid (EDTA), sodium fluoride (NaF), and saturation with thymol (extracted from Thymus vulgaris L.) in order to prevent contamination and bacterial growth. The solution is adjusted to a pH of 8.0. The solution can be used to collect samples and to maintain the samples at ambient temperature for an indefinite period for the later extraction of nucleic acids and PCR, cloning, sequencing, hybridization or mutagenesis with sufficient integrity and reliability.

### BACKGROUND

The present invention relates to the field of molecular biology, more particularly to methods, reagents and systems for preserving nucleic acids at an optimum quality (DNA, RNA, cDNA, mitDNA) in their cell of origin in solid tissues (organs) and liquids (body fluids) for an indefinite period of time and at ambient temperature.

Diagnostic analyses or tests based on nucleic acids (DNA or RNA) are becoming more frequent in clinical laboratories, hospitals, veterinarian clinics etc. These tests allow the genetic identification of any individual, and establish an individual's genetic relationship with their parents or siblings, (mother, father, son) or with individuals belonging to other populations (within breeds, species, genus, family or class). These tests also allows the detection of a mutation at a particular gene, the presence of a pathogen, or to predict a type of tumour.

Most of these tests are done indirectly by analyzing particular genetic markers. However, in the last few years, the use of Polymerase Chain Reaction or PCR (Mullis et al., U.S. Pat. Nos. 4,683,195, 4,683,202, 4,800,159 and 4,965,188) has allowed more specific, sensible and cost effective analysis from a molecular perspective.

To be able to perform these analyses, a good-quality nucleic acid is necessary. A good-quality nucleic acid is double stranded, not degraded nor fragmented. Many factors affect the quality of DNA samples (e.g., type of tissue maintained, type of additive used, length and form of transport to the laboratory) and the stability of the analyzed markers. Therefore, it is important to decide which DNA conservation method will be used before starting with a DNA bank.

There are many references which disclose DNA conservation methods that use no additives and are performed at ambient temperature: in amniotic liquid samples for 8 days (Casareale et al., 1992, PCR Methods Appl. Nov; 2(2): 149-53); in blood, methods vary from 24 h (Polakova et al., 1989, Bratisl. Lek. Listy. Nov; 90(11):844-7), to a maximum of four weeks (Joss & Do, 1997, J. Med. Microbiol. Jan; 46(1):92-6); in blood deposited and dried on a glass slide (Aggarwal, 1992, Pharmacotherapy, Aug; 22(8):954-60); on bucal wash maintained for 60-90 days at room temperature (Andrisin, 2002, Pharmacotherapy, Aug; 22(8):54-60). All these former references disclose negative results. Experiments done at higher temperatures, 23° C. during one week (Madisen et al., 1987, Am. J. Med. Genet. Jun; 27(2):379-90) or at 37° C. (Cushwa & Medrano, 1993, Biotechniques, 14(2):204-7), also produced a degraded nucleic acid.

When an additive is used, DNA stays in good condition after 72 weeks if the sample is mixed with phenol (Albarino & Romanowski, 1994, Mol. Cell Probes, Oct; 8(5):423-7), or with another additive called LST (Low Cost Storage and Transportation buffer), which maintains the DNA samples for a maximum of four weeks at room temperature. With LST, the DNA sample may also be frozen if more time is needed (Schultz, 1999, Am. J. Clin. Pathol., Jun; 111(6):748-52). Fukatsu (1999, Mol. Ecol., Nov; 8(11):1935-45) discloses DNA in good-quality after six months if the samples are conserved in acetone, ethanol, 2-propanol, and diethyl ether acetate.

There is also another form of conserving DNA in their cells of origin by maintaining blood on a piece of paper. There are a variety of commercially available cards for this purpose. Many references describe FTA cards with different results. For example, Natarajan et al. (Biotechniques, 2000, Dec; 29(6):1328-33) describes satisfactory, quality results with FTA cards, although it does not describe a specific time period of conservation. However, other references have shown that FTD cards exhibit a lower sensibility over time for the detection or presence of pathogenic DNA such as malaria (Zhong et al., 2001, J. Clin. Microbiol., Mar; 39(3): 1195-6). The same occurs with commercially available Isocode STIX cards.

Another reference discloses that blood on FTA cards maintains the DNA sample for extraction for over one year whenever the conservation temperature is −20° C., or for two to three months at room temperature (Natarajan et al., 2000, Biotechniques, Dec; 29(6): 1328-33).

Guthrie cards are paper cards in which blood from newborns is deposited with the aim of analyzing metabolic alterations. Following Makowski (Ann. Clin. Lab. Sci, 2000, Summer; 33(3):243-50), these cards can be a good source of DNA as they allow the conservation of blood for many years. Sierra Diagnostics uses a conservation method to maintain urine samples for many hours based on an amount of a divalent metal chelator selected from EDTA, EGTA or BAPTA at low molarities (in the range of from about 0.001 M to 0.1 M) that is mixed with lithium chloride, guanidine or sodium salicylate or sodium perchlorate, and sodium thiocyanate. This patented method (U.S. Pat. No. 6,458,546B1) maintains urine samples for DNA extraction for a maximum of ten days.

The most common method used to maintain DNA samples for long periods with no loss of DNA quality is to freeze the DNA at different temperatures: at −18° C. for between 7 days and 3 months, for six months at −70° C. while thawing many times (Naber, 1996, Diag. Mol. Pathol., Dec; 5(4):253-9), or for at least two months at −70° C. (Madisen et al., 1987, Am. J. Med. Genet., Jun; 27(2):379-90). The use of this last temperature is recommended to maintain tissues and blood and DNA is extracted only when there is a need (Holland et al., 2002, Mut. Res. 7702: 1-18).

Collection of blood samples in field conditions can only be performed with an anticoagulant, generally EDTAK₃ (Nielsen, 1985, Acta. Pathol. Microbiol. Immunol. Scand [C], Apr; 93(2):49-52) at room temperature, and a shortened time period for transport to the laboratory and extraction of the DNA. This time period can be much longer if the blood sample is refrigerated at 5° C. In these conditions, DNA extraction must be immediate upon arrival to the laboratory to avoid degradation, or the sample must be frozen to maintain the integrity of the nucleic acids. Because of this factor, in the laboratories, there is a tendency to extract the nucleic acid of interest at reception to avoid degradation and ensure analysis accuracy. This need for immediate extraction causes an uncertainty in the laboratory because it leads to an unknown number of DNA samples.

The addition of an antiseptic reagent in the collection solution lengthens the half life of the nucleic acids in their cell of origin at ambient temperature by avoiding bacterial growth and activity of nucleases that degrade nucleic acids. However, no reference discloses the addition of an antiseptic agent. Thymol is an antiseptic agent and disinfectant that destroys the vitality of live ferments, annulates, and enzymes present in the medium, and prevents or reduces putrefaction. A small quantity of thymol added to albumin, milk, and gelatin preserves a solution of these compounds for months (Harvey Wickes Felter, MD, & John uri Lloyd, Phr. M., Ph.D.; 1898).

Thymol has been used as a disinfectant that is dissolved in water (Giraldes, M), as a basis to do inhalations for respiratory affections (Bouillhon and Paquet), to treat diphtheria and other illnesses produced by microorganisms, such as articular rheuma, typhus, phthisis and pielitis, as a helminthic agent, as well as an agent to treat cutaneous problems such as eczema, psoriasis or burns. Currently, thymol is used almost exclusively in collutory solutions for the treatment of gingivitis, as inhalations mixed with camphor, for topic use as liniments, as a topic anaesthetic (http:/www.uhe.com/thymol.htm), and in general as an antiseptic and antioxidant component in medicine or industry or as a stabiliser of different anaesthetics such as halothane (Szentandrassy et al., “Effect of thymol on kinetic properties of Ca and K currents in rat skeletal muscle,” 2003, BMC Pharm. 3:9). Thymol is also currently used to treat varroasis for bees, being a less harmful synthetic acaricide than oxalic acid (Imdorf, A. et al., “Toxicity of thymol, camphor, menthol and eucalyptol in Varroa jacobsoni and Apis mellifera in laboratory tests,” 1995, Apidologie 26:27-31).

Often, DNA sample collection is performed in places that are far away from the laboratories where the samples will be processed. The samples are also often collected under difficult conditions (such as high temperatures or places where it is difficult to use appropriate refrigeration). Therefore, it is necessary to have available an additive which, once mixed with any type of tissue (such as an organ fragment, a vaginal swab etc.) or any fluid (blood, semen, amniotic liquid etc.), maintains DNA samples in such conditions that the nucleic acids in the cells remain in a constant condition for an indefinite period, even at ambient temperature.

### SUMMARY

The present invention relates generally to a solution that maintains a sample collected from an individual in such conditions that the quality of nucleic acids extracted later will not be affected, guaranteeing the nucleic acids' integrity independently of the time elapsed between the sample collection and the extraction of the nucleic acid and the temperature at which the sample has been collected. It also relates generally to a solution for the indefinite maintenance of nucleic acids in their cell of origin that maintains any biological sample obtained from an individual eukaryote or a prokaryote at ambient temperature or at a temperature at or above 5° C.

In one embodiment, this maintenance is made possible by mixing the sample with a solution containing EDTA, NaF and thymol, and equilibrating the mixture to a pH of 8.0. EDTA is a potent divalent metal chelator which acts by eliminating cations that are components of metal-dependent enzymes that degrade nucleic acids (e.g., DNAses). The presence of EDTA inactivates these enzymes.

In an embodiment, NaF is another component present in the solution. NaF is another chelator which helps to inactivate enzymes such as ligases, polymerases, exonucleases, kinases, nucleases. Its presence improves the chelating capability of EDTA.

In one embodiment, the solution is also comprised of thymol, an essential oil extracted from thyme (Thymus vulgaris L.) that is an antiseptic, antifungal and antihelminthic. Thymol (or thyme camphor), also called isopropyl-metacresol, 6-isopropyl-m-creol, 3-hydroxy-p-cymene, isopropyl cresol, or 2-isopropyl-5-methylphenol, has a molecular formula of C₁₀H₁₄O, and is a crystalline phenol that is obtained from the volatile oils of Thymus vulgaris, (Linn) (N. O. Labiatae), Monarda punctata, (Linn) (N. O. Labiatae), Carum copticum, (Benth & Hook, f.) (N. O. Umbelliferae), and other plants.

Thymol has the same mode of action as phenol. However, due to its insolubility in body fluids, thymol is absorbed slower than phenol. Moreover, thymol is less irritating, and its germicide action is higher than that of phenol. Thymol is a more potent bactericide than other phenols (Juven et al., “Factors that interact with the antibacterial action of thyme essential oil and its active constituents,” Appl. Bacteriol, 1994, Jun; 76(6):626-31). Thymol acts on cellular membranes producing a lack of membrane selective permeability by changing its physical properties. This action on the membranes is common to all phenols and is due to the coexistence of an hydrophilic and a lipophilic region. Thymol produces insoluble proteinates due to the enzymatic inactivation and the protein denaturation, and is the factor which avoids degradation of nucleic acids through the presence of enzymes and thus allows their integrity through long periods of time. Methyl group and isopropil chain improves the antiseptic activity in a more efficient way than phenol as thymol toxicity is lower than that of the phenol.

The product is performed by mixing in a solution an amount of a divalent metals chelator EDTA (Ethylen-diamine-tetraacetic acid) at a molarity varying between 0.39 M and 0.56 M in its trisodium form; a component improving chelating capacity of EDTA which is Sodium Fluoride (NaF) which varies between 0.33 M and 0.48 M and the saturation of this solution with thymol (less than 0.0001 M) to avoid microorganisms growth in the sample while the latter is maintained at temperatures equal or higher then +5° C. and its pH equilibration to a value of 8.

The solution acts on the cellular membrane by producing changes in the physical properties which makes the membrane loosely permeable. The solution also inactivates degrading enzymes by chelating the enzymes' cations, and contributes to protein denaturation thereby producing insoluble proteinates.

The solution, when added to any tissue, body fluid, tumour tissue, skin, bone marrow, feather, hair, blood, blood serum, amniotic liquid, egg, semen, saliva, vaginal fluid, sweat, preserve the sample and maintains nucleic acids in the sample for an indefinite period of time and at any temperature greater than +5° C. This solution avoids the enzymatic destruction of the nucleic acids and avoids bacterial, fungal or helminthic growth in the solution that is not due to the sample and which appears together with enzymes that degrade nucleic acids.

According to an embodiment of the present invention, it is necessary to submerge the sample in a solution containing EDTA (ethylene-diamine-tetraacetic acid) in its trisodium form and at a concentration at or greater than 0.39 M, and sodium fluoride (NaF) at a concentration at or greater than 0.33 M. According to one embodiment, the solution must also be saturated with thymol and equilibrated to a pH of 8.0. Moreover, the nucleic acids referred to in the present invention may be any nucleic acid, including but not limited to, deoxyribonucleic acid (DNA), ribonucleic acid (RNA), coding deoxyribonucleic acid (cDNA) and mitochondrial deoxyribonucleic acid (mitDNA) contained in any eukaryotic or prokaryotic cell.

Mixing the solution of the present invention with a biologic sample preserves the solution in such conditions that the nucleic acid quality extracted a few hours or years later will not be affected, guaranteeing its integrity independently of the time elapsed between obtaining the sample and nucleic acid extraction. The method of mixing the solution with a biological sample also guarantees that the processes which will be performed on the nucleic acids, including but not limited to, amplification through PCR, sequencing, cloning, hybridization, mutagenesis or similar techniques, will be of high quality. The solution of the present invention can be prepared in a liquid, solid or micronized form, and allows the preservation of nucleic acids for an indefinite period of time in tissue and body fluid samples of eukaryotic and prokaryotic cells.

In one embodiment, the ability of the described procedure to maintain a nucleic acid in its cell of origin in such conditions that any type of analysis can be successfully performed independently of the time elapsed since sampling has been performed can be observed by comparing samples which are mixed with the solution of the present invention and samples which are not mixed with the solution of the present invention. Both mixtures should also be preserved at +5° C. The difference between the samples can be observed through the use of the absorbance measure given by the nucleic acids after their extraction at different times from sampling as a quality parameter. As shown in FIG. 1, independently of the time elapsed, samples mixed with the solution of the present invention reach equivalent quantities of DNA, but samples not maintained in the solution of the present invention will show increased amounts of DNA as a consequence of bacterial growth and will decay to zero after a few weeks.

### BRIEF DESCRIPTION OF THE DRAWINGS

FIG. 1 is a comparison of absorbance of DNA extracted from samples preserved in the solution of the present invention and DNA extracted from samples that were not preserved in the solution of the present invention. Both samples were maintained at +5° C. for 8 months.

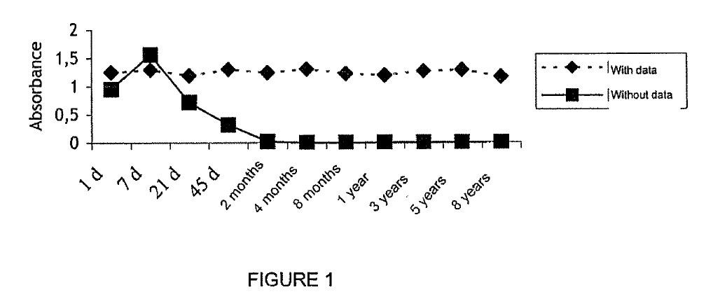

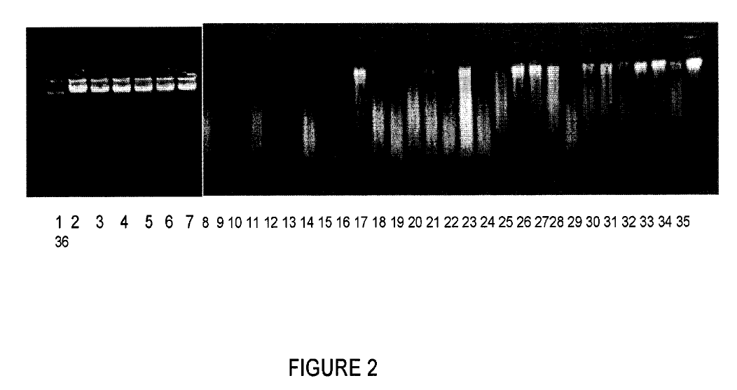

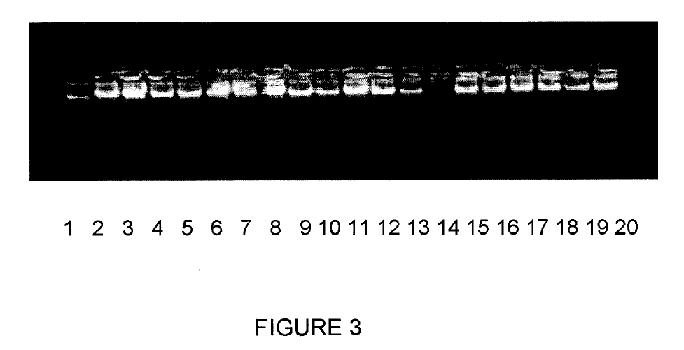

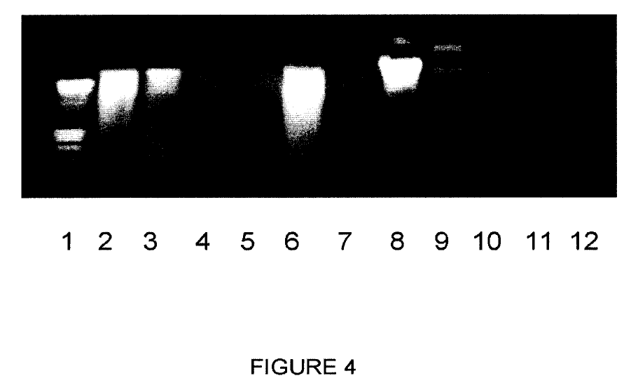

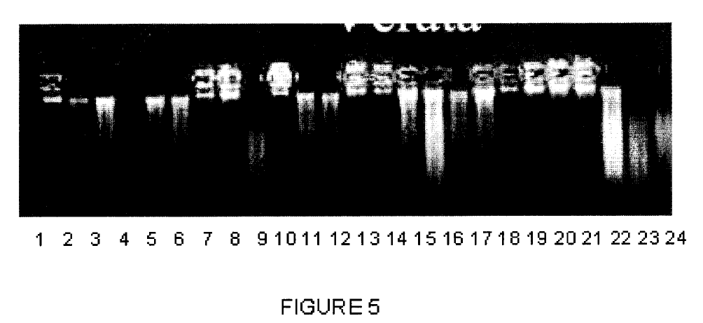

FIG. 2 is a comparison of absorbance between DNA from blood, extracted 48 hours and 8 months after being preserved in a solution which contains EDTA, NaF and thymol as described herein, or preserved in EDTA K₃ and maintained at +5° C., or preserved in EDTA K₃ at −20° C. The other sample of blood was not preserved in any of these described solutions.
1: λ marker
2-7: blood mixed with the invention
8-22: blood +EDTA K₃ refrigerated
23-36: blood +EDTA K₃ frozen

FIG. 3 shows the absorbance results when DNA is extracted (blood with alkaline lysis and tissue with phenol chlorophorm) after maintaining the samples for 8 months at +5° C. in a solution that contains EDTA, NaF and thymol at pH 8.0.
1: λ marker
2-6: blood
7-11: muscle
12-15: hair
16-20: cartilage

FIG. 4 shows the absorbance results of DNA extracted from different samples conserved without any additive for 48 hours (1-7) and without additive and frozen (8-12), visualised in a 0.6% agarose gel.
1: λ marker
2-3: blood
4-5: muscle
6: hair
7: cartilage
8-9: blood
10: muscle
11: hair
12: cartilage

FIG. 5 shows the absorbance results of DNA extracted from different samples (blood, muscle, cartilage, hair) that were preserved for 8 months in EDTA K₃ at −20° C.
1: λ marker
2-8: blood
9-14: muscle
15-21: cartilage
22-24: hair

### DETAILED DESCRIPTION

Different experiments were performed with different biological tissues, different types of treatments, including the solution of the present invention, and different time periods since sampling.

In one embodiment, biological material included, but is not limited to, blood, ear cartilage, hair and muscle. For each tissue, 50 animals were analyzed, and five replicates were analyzed for each animal analyzed.

Sample conservation was performed at three temperatures, room temperature, refrigeration between 5° C. and 7° C., and frozen at −20° C., with three different treatments, no additive, with EDTA K₃ and with the solution of the present invention.

Samples were maintained at a minimum of 48 hours and a maximum of eight months.

### EXAMPLE 1

A method to extract genomic DNA is presented from blood samples maintained at room temperature and preserved in the solution of the present invention.

The solution consisted of 1 gram of EDTA trisodium, 100 milligrams of NaF, and 0.1 milligrams of thymol at a pH of 8.0. The solution was introduced to a 5 ml vacuum tube. Five ml of blood were sampled by venopunction, and the tube was thoroughly mixed by inversion. The tube was maintained at room temperature for one week, and then maintained at 5° C. indefinitely. The tubes were then maintained at room temperature for 16 hours before extracting DNA in order to get a good homogenization of blood and solution.

To extract high quantities of DNA (more then 5 μg of DNA), an aliquot of between 1 ml and 3 ml of blood plus solution were taken. The same volume of Tris ClH 10 mM+EDTA 10 mM was added, mixed and centrifuged for three minutes. The supernatant was discarded without touching the pellet. This step was repeated by adding Tris ClH 10 mM+EDTA 1 mM. After discarding the supernatant, the pellet was incubated in a lysis solution consisting of NaCl 0.1 M and EDTA 25 mM, 12.5 μl SDS 10%, 0.1 mg Proteinase K at 37° C. for two or more hours. Next, phenol+chlorophorm+isoamilic was added in a proportion of 25:24:1. After centrifugation for 10 minutes, the supernatant was moved to a new tube and DNA was precipitated with 100% ethanol after addition of 40 μl NaAc 3 M at a pH of 5.2 (See FIGS. 2 and 3).

### EXAMPLE 2

In this example, tissue samples were comprised of 500 milligrams of muscle and 50 milligrams of cartilage. The different tissues were mixed with a solution containing NaCl 0.14 M, MgAc 1.5 mM, ClK 5 mM, and 1% SDS, and a manual homogenisation treatment for one minute was performed. Phenol-chlorophorm-isoamilic was then added in a proportion 25:24:1. After 10 minutes of shaking, centrifugation was performed for five minutes and the supernatant (aqueous phase) was moved to a new tube. DNA was then precipitated with 100% ethanol after addition of 40 μl 3M NaAc at a pH of 5.2 (see FIG. 4).

### EXAMPLE 3

A genomic DNA extraction method is presented for a blood sample contained in the solution of the present invention. The solution consists of one gram of EDTA trisodium, 100 milligrams of NaF, and 0.1 milligrams of thymol at a pH of 8.0. The solution is then introduced to a five ml vacuum tube.

Five ml of blood is then obtained by venopunction, and the tube is mixed by inversion. The tube is maintained at ambient temperature indefinitely. On arrival to the extraction laboratory, maintenance at +5° C. is recommended. Before extracting DNA, tubes are submitted for 16 hours to room temperature in order to get a homogenization of blood and solution. 50 μl (for amplification of one or two genes), or 100 μl (for an hybridisation test) of blood is taken with a micropipette and passed to a new tube. 100 μl of distilled H₂O is then added. After mixing and 3 minutes of centrifugation, the supernatant is discarded. This step is repeated twice. 100 μl of NaOH 0.25 M is then added and this mixture is incubated for 15 minutes. 100 μl of ClH 0.25 M and 100 μl Tris ClH 0.1 M is then added and the solution is equilibrated to a pH of 8.5. This mixture is then centrifuged, and 2 to 5 μl of the supernatant is taken for PCR amplification. (See FIG. 2 and 3).

For the purposes of promoting an understanding of the principles of the invention, reference has been made to the embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

1. A solution for the indefinite maintenance of nucleic acids in their cell of origin comprised of the following:
a. a concentration of EDTA (Ethylen-diamine-tetraacetic acid) at a molarity from 0.39 M and 0.56 M in its trisodium form (Na₃);
b. a concentration of sodium fluoride (NaF) at a molarity from 0.33 M to 0.48 M;
c. a saturating amount of thymol at a molarity of about 0.0001 M, wherein said thymol is extracted from Thymus vulgaris L.; and
d. an equilibrating amount of ClH that equilibrates said solution to a pH 8.0.

2. The solution according to claim 1, wherein an amount of EDTA in a one liter solution is from between 140 grams and 200 grams.

3. The solution according to claim 1, wherein an amount of NaF in a one liter solution is from between 14 grams and 20 grams.

4. The solution according to claim 1, wherein the amount of thymol in a one liter solution is from between 5 mg. and 10 mg.

5. (canceled)

6. The solution according to claim 1, wherein said solution is maintained at a temperature at or above +5° C.

7. (canceled)

8. The solution according to claim 1, wherein the solution is one of an aqueous, solid or micronised solution.

9. A method for maintaining indefinitely one or more nucleic acids in their cell of origin comprised of the following steps:
preparing a solution that is comprised of a concentration of EDTA (Ethylen-diamine-tetraacetic acid) at a molarity from 0.39 M to 0.56 M in its trisodium form (Na₃), a concentration of sodium fluoride (NaF) at a molarity from 0.33 M to 0.48 M, a saturating amount of thymol at a molarity of about 0.0001 M, wherein said thymol is extracted from Thymus vulgaris L., and an equilibrating amount of ClH that equilibrates said solution to a pH 8.0;
adding the solution to a biological sample obtained from an individual eukaryote or prokaryote; and
maintaining the solution and biological sample at a temperature at or above +5° C.

10. The method according to claim 9, wherein an amount of EDTA in a one liter solution is from between 140 grams and 200 grams.

11. The method according to claim 9, wherein an amount of NaF in a one liter solution is from between 14 grams and 20 grams.

12. The method according to claim 9, wherein the amount of thymol in a one liter solution is from between 5 grams and 10 grams.

13. The method according to claim 9, wherein the pH of the solution is equal to 8.0.

14. The method according to claim 9, wherein said solution is one of an aqueous, solid or micronised solution.

15. The method according to claim 9, wherein the biological sample is comprised of one or more of the following: blood, an organ or a fragment of an organ, a healthy or a tumor or an inflamed tissue, skin, feather, bone marrow, egg, blood serum, amniotic liquid, semen, saliva, vaginal fluid, sweat, feces, urine, hair or a cell culture.
